(19) United States
(12) Patent Application Publication
(10) Pub. No.: US 2019/0099071 A1
(43) Pub. Date: Apr. 4, 2019

(54) Devices and Method for Generating A Stimulus to Evaluate Ocular Sensitivity

(71) Applicant: Brien Holden Vision Institute, Sydney, NSW (AU)

(72) Inventor: Klaus Ehrmann, Queenscliff, NSW (AU)

(21) Appl. No.: 16/086,950

(22) PCT Filed: Mar. 24, 2016
(86) PCT No.: PCT/AU2016/050216

§ 371 Date: Sep. 20, 2018

(51) Int. Cl. A61B 3/00; G02B 27/00; A61B 3/02; A61B 3/13

(52) U.S. Cl. CPC A61B 3/0083; A61B 3/132; A61B 3/02; G02B 27/0093

(57) ABSTRACT

A device for generating a stimulus in the form of at least one liquid droplet to evaluate ocular sensitivity, the device comprising a light source configured to illuminate an eye of the subject; a liquid reservoir configured to store a liquid; and a nozzle in fluid communication with the liquid reservoir and configured to deliver at least one liquid droplet to an eye of a subject. Delivery of the at least one liquid droplet to the eye of the subject provides a stimulus to the ocular surface of the subject's eye and enables the evaluation of the ocular sensitivity of the subject's eye.

### FIELD OF THE DISCLOSURE

This disclosure is related to devices and methods for generating a stimulus to evaluate ocular sensitivity. For example, this disclosure is related to devices and method for generating mechanical, chemical, and/or thermal stimulus, applying the stimulus (e.g., a liquid droplet) to the ocular surface of a subject and evaluating the ocular sensitivity of the subject.

### BACKGROUND

Ocular sensitive of a subject may be evaluated in numerous manners. Devices used for such an evaluation are generally referred to as an aesthesiometer (or esthesiometer) and typically use a filament or controlled pulse of air to generate a stimulus on the cornea. In the case of the filament based aesthesiometer (e.g., a Cochet Bonnet filament aesthesiometer), a nylon monofilament may be utilized. The filament may have a constant diameter with varying length. Depending on the length of the filament, the device may exert more or less pressure on the cornea. In a similar operation, the aesthesiometers based on the use of pressurized air (e.g., a Belmonte gas aesthesiometer) may vary the pulse of pressurized air to stimulate the cornea.

These devices are generally used to determine at what threshold the subject responds to the stimulus being provided. For a variety of reasons, these methods/devices are not capable of providing a stimulus to precise locations of cornea and have suffered from poor repeatability. In addition, these devices generally only provide a mechanical stimulus and the effectiveness of their operation is highly dependent on the skill of the operator.

Also, it should be noted that while the Cochet Bonnet filament aesthesiometers have been used in practice, the Belmonte gas aesthesiometer were briefly available commercially but were not successful because the devices did not operate in a satisfactory manner.

Contact lens manufactures have an interest in improving the on-eye comfort of their products and accurate measurement of corneal sensitivity is thought to assist researchers to investigate the underlying cause of comfort related dropout. This information may help with the development of better performing lenses. Additionally, ocular comfort is an issue in many eye related diseases and conditions, including, for example, dry eye, Meibomian Gland Dysfunction (MGD), Lasik, etc. In addition, contact lens providers have an interest in improving the on-eye comfort of the contact lens they provide and accurate measurement of corneal sensitivity may assist the providers in ameliorating the discomfort that some individuals experience with wearing contact lens. This information may help with providing a better selection of the contact lens for a particular individual. The location within the eye where discomfort originates may also be of interest to contact lens manufactures and/or contact lens providers. Knowing which locations and/or tissues (i.e. central/peripheral cornea, conjunctiva, eyelids or lid margins) are more sensitive may assist in helping individual patients to improve their symptoms and/or provide the contact lens industry with more specific targets to improve their products.

Accordingly, there is a need for an aesthesiometer that is capable of assisting with one or more of the shortcomings of the existing devices by, for example, providing a stimulus to a precise location on the cornea and/or is providing different combinations of stimulus including one or more of a chemical, mechanical, and/or thermal stimulus.

### SUMMARY

In exemplary embodiments, the devices and/or methods may benefit from better reliability than current devices and/or methods. In exemplary embodiments, the devices and/or methods may benefit from better repeatability than existing devices and/or methods. In exemplary embodiments, the device and/or method may benefit from providing more than a mechanical stimulus to the subject. In exemplary embodiments, the devices and/or methods may benefit from being easier to use (and/or require reduced levels of training) than existing devices and/or methods. In exemplary embodiments, the devices may benefit from being relatively small in size and/or attachable to an existing instrument (e.g., a slit lamp).

In exemplary embodiments, various combinations or one or more of the above benefits may enable the devices and/or methods to obtain more acceptability in a commercial setting. For example, more ophthalmologists may utilize the device in their practice.

Exemplary embodiments may provide a device for generating a stimulus in the form of at least one liquid droplet to evaluate ocular sensitivity, the device comprising: a light source configured to illuminate an eye of the subject; a liquid reservoir configured to store a liquid; a nozzle in fluid communication with the liquid reservoir and configured to deliver at least one liquid droplet to an eye of a subject; wherein delivery of the at least one liquid droplet to the eye of the subject provides a stimulus to the ocular surface of the subject's eye and enables the evaluation of the ocular sensitivity of the subject's eye.

In exemplary embodiments, the term liquid droplet should be readily understood to mean a volume of liquid which tends over time towards a substantially spherical shape. For example, depending on the length of the stimulus, the droplet may be substantially spherical when it leaves the nozzle (short time) or more elongated (longer time). However, droplets of other shapes may be used as well.

In exemplary embodiments, a slit lamp device (or at least slit lamp functionality) configured to illuminate the eye of the subject and provide a view (e.g., a magnified view) of the subject's eye may be provided.

In exemplary embodiments, circuitry configured to adjust various parameters (e.g., pressure, pulse duration, pulse frequency, pulse delay, etc.) to generate the at least one liquid droplet such that it possesses the desired parameters (e.g., size, velocity, etc) may be provided.

In exemplary embodiments, circuitry of the device is configured to adjust one or more of the following parameters: pressure, pulse duration, pulse frequency and pulse delay to generate the at least one liquid droplet such that it possesses the desired size and/or velocity.

In exemplary embodiments, the device may further comprise a temperature controller for controlling the temperature of the at least one liquid droplet delivered to the subject.

In exemplary embodiments, the device may further comprise a heating element (or cooling element) for altering the temperature of the liquid.

In exemplary embodiments, the device may further comprise a heating element (or cooling element) located within the valve assembly.

In exemplary embodiments, the liquid droplet may create a mechanical, chemical, and/or thermal stimulus.

In exemplary embodiments, the liquid may be tear-like and/or may be warmed up to substantially the same temperature as the eye of the subject.

In exemplary embodiments, the volume of one or more droplets and/or its velocity may be adjusted to adjust the stimulus.

In exemplary embodiments, a sub-mechanical threshold setting may be used and the liquid may be modified to make it increasingly acidic or alkaline i.e., use of a soap and/or concentrated saline solutions, etc.

In exemplary embodiments, the liquid droplet may be heated or cooled while keeping the mechanical stimulation at a sub-threshold level.

In exemplary embodiments, the device may be configured to apply the liquid droplet to a precise, predetermined location on the ocular surface of the subject's eye.

In exemplary embodiments, the device may be considered non-invasive.

In exemplary embodiments, the liquid may be selected or treated so as not to harm the subject's eye (e.g., sterile, not causing infections, etc.).

In exemplary embodiments, the device may be configured to provide repeated stimulus on the same or substantially the same location.

In exemplary embodiments, the device may further comprise two or more nozzles and the two or more nozzles may be configured to provide various combinations of substantially simultaneous, simultaneous or alternate stimulus.

In exemplary embodiments, the device may be configured to test the spatial resolution of the sensory system by having two droplets contact the surface of the subject's cornea simultaneously (or substantially simultaneously) with adjustable lateral separation.

In exemplary embodiments, the device may be configured such that two droplets are used to make comparative measurements. For example, the device may be configured to stimulate the left and right eye simultaneously (or substantially simultaneously) to determine if there is a difference in sensitivity between the eyes. Alternatively, the device may be configured to stimulate central and peripheral cornea or cornea and lid margin in a simultaneous (or substantially simultaneous) manner to determine which region is more sensitive. In exemplary embodiments, comparative measurements may be more reliable than absolute measurements.

In exemplary embodiments, the device may be configured to include presentation of a bright, high contrast image to the contralateral eye for the subject to concentrate on.

In exemplary embodiments, the device may be configured to reduce or minimize the volume of the droplet or increase the velocity to achieve perceived stimulation.

In exemplary embodiments, the device may be configured to switch off illumination shortly before the droplet is projected to eliminate (or minimize or reduce) the potential that subjects may respond to a visual effect rather than the mechanical, chemical, and/or thermal stimulation of the ocular surface (e.g., in a randomized manner to assist with the masking of the stimulus).

In exemplary embodiments, the device may further comprise a trigger (e.g., a button) to enable the operator to administer the liquid droplet to the subject.

In exemplary embodiments, the device may further comprise a feedback interface (e.g. a button) configured to enable the patient to acknowledge whether they were able to perceive the liquid droplet.

In exemplary embodiments, the liquid may comprise nanoparticles suspended in the liquid or mixed with the liquid prior to delivery and selected to achieve a specific goal (e.g., size, color, active coating, etc.).

In exemplary embodiments, the liquid may comprise nanoparticles suspended in the liquid or mixed with the liquid prior to delivery and selected to achieve one or more of the following goals: size, color and active coating.

In exemplary embodiments, the device may be configured such that a plurality of droplets of equal size and velocity may be generated in rapid sequence (e.g., 1, 2, 3, or 4 kHz) to increase the stimulus strength of the liquid.

In exemplary embodiments, the device may be configured such that the delay time between two or more repeated stimuli may be varied.

In exemplary embodiments, the device may be configured such that responsiveness may be evaluated by having two or more valves that deliver droplets simultaneously (or substantially simultaneously) and the lateral separation of the droplets may be varied (or alternatively with one valve that moves quickly between two positions).

In exemplary embodiments, the device may be configured to avoid delivering too many large liquid droplets at rapid sequence, thereby reducing or minimizing disturbance to the integrity of the normal tearfilm.

In exemplary embodiments, the liquid may be degassed to prevent, minimize and/or reduce air bubbles in the system.

In exemplary embodiments, the device may be configured to slightly vary the actual time-point of stimulation with respect to other, earlier stimulation or in relation to the diming of illumination.

In exemplary embodiments, the stimulus may be synchronized or substantially synchronized with the subject's blink.

In exemplary embodiments, optical or acoustical signals may be used to trigger a blink at a pre-determined time period prior (or after) delivering the liquid droplet. For example, the optical signal may be generated by an LED or other desirable lighting source. For example, the acoustical signal may be generated by a speaker or other desirable sound source.

In exemplary embodiments, the device may be configured to monitor the blink of the subject and to deliver the droplet after a certain delay time.

In exemplary embodiments, the device may be configured such that the working distance between the nozzle and the ocular surface may be about 10, 20, 30, 40, 50, or 60 mm.

In exemplary embodiments, the device may be configured such that the working distance between the nozzle and the ocular surface may be between 5 to 70 mm, 10 to 40 mm, 10 to 30 mm, 20 to 50 mm, or 30 to 60 mm.

In exemplary embodiments, active pressure generating devices (used with our without a valve) may be used to eject a liquid droplet (e.g. similar to bubble jet technology or piezo activated printing head technology).

In exemplary embodiments, the device may be configured such that a chemical stimulus may be generated by utilizing two or more ejectors aimed at the same spot on the ocular surface—one ejector configured to deploy a chemical stimulant and the other injector configured deploy plain water or a neutralizing liquid, in a predefined ratio. Similarly, in exemplary embodiments, the device may be configured such that two or more ejectors may be utilized to modify the temperature of the liquid delivered to the subject. For example, the two ejectors may hold liquids at different temperatures which may be delivered to the subject in varying ratios to control the temperature of the liquid.

Exemplary embodiments may provide a method for evaluating ocular sensitivity, the method comprising: storing a liquid in a liquid reservoir; transmitting the liquid from the liquid reservoir to a nozzle; generating at least one liquid droplet; and delivering the at least one liquid droplet to the ocular surface of a subject's eye; wherein the delivery of the at least one liquid droplet to the eye of the subject provides a stimulus to the ocular surface of the subject's eye and enables the evaluation of the ocular sensitivity of the subject's eye.

In exemplary embodiments, the method may further comprise providing a light source configured to illuminate an eye of the subject;

In exemplary embodiments, the method may further comprise adjusting various parameters (e.g., pressure, pulse duration, pulse frequency, pulse delay, etc.) to generate the at least one liquid droplet such that it possesses the desired parameters (e.g., size, velocity, etc).

In exemplary embodiments, the method may further comprise adjusting one or more of the following parameters: pressure, pulse duration, pulse frequency and pulse delay to generate the at least one liquid droplet such that it possesses the desired size and/or velocity.

In exemplary embodiments, the method may further comprise controlling the temperature of the at least one liquid droplet delivered to the subject.

In exemplary embodiments, the method may further comprise heating (or cooling) the liquid.

In exemplary embodiments, the liquid may be heated within the valve assembly.

In exemplary embodiments, the liquid droplet may create a mechanical, chemical, and/or thermal stimulus.

In exemplary embodiments, the liquid may be tear-like and/or may be warmed up to substantially the same temperature as the eye of the subject.

In exemplary embodiments, the method may further comprise adjusting the volume and/or velocity of the liquid droplets to adjust the stimulus.

In exemplary embodiments, the method may further comprise reducing the delivery of the liquid droplet to a sub-mechanical threshold and modifying the liquid to make it increasingly acidic or alkaline i.e., use of a soap and/or concentrated saline solutions, etc.

In exemplary embodiments, the method may further comprise heating or cooling the liquid droplet while keeping the mechanical stimulation at a sub-threshold level.

In exemplary embodiments, the method may further comprise applying the liquid droplet to a precise, predetermined location on the ocular surface of the subject's eye.

In exemplary embodiments, the method may be considered non-invasive.

In exemplary embodiments, the method may further comprise providing repeated stimulus on the same, or substantially the same, location of the ocular surface.

In exemplary embodiments, two or more nozzles may be provided and the two or more nozzles may be configured to provide various combinations of simultaneous or alternate stimulus.

In exemplary embodiments, the method may further comprise testing the spatial resolution of the sensory system by having two droplets contact the surface of the subject's cornea simultaneously (or substantially simultaneously) with adjustable lateral separation.

In exemplary embodiments, the method may further comprise presenting a bright, high contrast image to the contralateral eye for the subject to concentrate on.

In exemplary embodiments, the method may further comprise reducing/minimizing the volume of the droplet or increase the velocity to achieve perceived stimulation.

In exemplary embodiments, the method may further comprise switching off the illumination shortly before the droplet is projected (e.g., in a randomized manner to assist with the masking of the stimulus).

In exemplary embodiments, the method may further comprise enabling the operator to administer the liquid droplet to the subject.

In exemplary embodiments, the method may further comprise enabling the subject to acknowledge whether they were able perceive the liquid droplet.

In exemplary embodiments, the method may further comprise suspending nanoparticles in the liquid or mixing the nanoparticles with the liquid prior to delivery to achieve a specific goal (e.g., size color, active coating, etc.).

In exemplary embodiments, the method may further comprise generating a plurality of droplets of equal size and velocity in rapid sequence (e.g., 1, 2, 3, or 4 kHz) to increase the stimulus strength of the liquid.

In exemplary embodiments, the method may further comprise varying the delay time between two or more repeated stimuli.

In exemplary embodiments, the method may further comprise evaluating responsiveness by having two or more valves that deliver droplets simultaneously such that the lateral separation of the droplets may be varied (or alternatively with one valve that moves quickly between two positions).

In exemplary embodiments, the method may further comprise avoiding delivering too many large liquid droplets at rapid sequence.

In exemplary embodiments, the method may further comprise degassing the liquid to prevent, minimize, and/or reduce air bubbles in the system.

In exemplary embodiments, the method may further comprise varying the actual time-point of stimulation with respect to other, earlier stimulation or in relation to the diming of illumination.

In exemplary embodiments, the stimulus may be synchronized or substantially synchronized with the subject's blink.

In exemplary embodiments, the method may further comprise triggering a blink using optical or acoustical signals at a pre-determined time period prior (or after) delivering the liquid droplet.

In exemplary embodiments, the method may further comprise monitoring the blink of the subject and delivering the droplet after a certain delay time.

In exemplary embodiments, the working distance between the nozzle and the ocular surface may be about 10, 20, 30, 40, 50, or 60 mm.

In exemplary embodiments, the working distance between the nozzle and the ocular surface may be between 5 to 70 mm, 10 to 40 mm, 10 to 30 mm, 20 to 50 mm, or 30 to 60 mm.

In exemplary embodiments, the method may further comprise generating a chemical stimulus by utilizing two ejectors aimed at the same or substantially the same spot on the ocular surface—one ejector configured to deploy a chemical stimulant and the other injector configured deploy plain water or a neutralizing liquid, in a predefined ratio.

### DESCRIPTION OF THE DRAWINGS

Notwithstanding other forms which may fall within the scope of the disclosure as set forth herein, specific embodiments will now be described by way of example and with reference to the accompanying drawings in which:

FIG. 1 is an exemplary embodiment of a liquid jet aesthesiometer capable of supplying a stimulus in the form of a liquid droplet to the ocular surface of a subject;

FIG. 2 is block diagram of an exemplary embodiment of a liquid jet aesthesiometer capable of supplying a stimulus in the form of a liquid droplet to the ocular surface of a subject;

FIG. 3 is an illustration of a control panel for use by an operator for controlling an exemplary liquid jet aesthesiometer; and

FIG. 4 is an exemplary embodiment of a valve assembly capable of generating and delivering the liquid droplet to the ocular surface of the subject.

### DETAILED DESCRIPTION

Exemplary embodiments of the disclosure relate to systems, methods, and/or devices for generating a stimulus to evaluate ocular sensitivity.

In exemplary embodiments, the devices and methods for generating the stimulus may utilize a liquid droplet to create a mechanical, chemical, and/or thermal stimulus, apply the stimulus to the ocular surface of a subject and evaluate the ocular sensitivity of the subject

In exemplary embodiments, the device may be utilized in a recursive step (e.g., staircase) approach to determine a sensation threshold of the subject. For example, beginning with a low threshold value, the stimulus strength may be increased in pre-determined steps until there is a positive response from the subject. At this point, the stimulus may be reduced by several steps and then increased again. The step size may be the same or it may be different (e.g., larger or smaller). This process may be repeated until several (e.g., 2, 3, 4, 5, or 6) reversals have been achieved. In exemplary embodiments, the average of the threshold values may be calculated and used as a threshold value for the patient.

In exemplary embodiments, the methods may also enable the patient to provide a subjective strength rating of the stimulus. For example, the while maintaining the strength of the stimulus constant, different subjects could be ask to rate the stimulus on a predefined scale (e.g., 1-10, etc.). However, as may be readily understood, such a rating is subjective and may lead to more variability within the results.

In exemplary embodiments, the device may generate a mechanical, chemical or thermal stimulus to the ocular/lid surface to evaluate ocular sensitivity. The device may be capable of creating a fine liquid droplet and propelling the droplet under controlled conditions onto the ocular surface to provide the stimulus and elicit a response from the subject. In exemplary embodiments, a range of different variables may be adjusted to change the type and/or strength of the stimulus. For example, in embodiments utilizing mechanical stimulation, the liquid may be tear-like and/or may be warmed up to substantially the same temperature as the eye of the subject. In exemplary embodiments, the stimulus strength may be varied by e.g., adjusting the volume of each droplet and/or its velocity. In embodiments utilizing chemical stimulation, a sub-mechanical threshold setting may be used and the liquid may be modified to make it increasingly acidic or alkaline i.e., use of a soap and/or concentrated saline solutions, lachrymatory agents such as capsaicin, alcohols of various concentrations etc. In embodiments utilizing thermal stimulation, the device may cool and/or heat the liquid droplet while possibly keeping the mechanical stimulation at a sub-threshold level.

In exemplary embodiments, the device may be a standalone device. In exemplary embodiments, the device may be integrated with other well known devices. In exemplary embodiments, the device may be a supplement to an existing device. For example, in exemplary embodiments, the device may be a supplemental device added to a slit lamp.

In exemplary embodiments, the device may be configured to apply the stimulus to a precise, predetermined location on the ocular surface of the subject's eye. In exemplary embodiments, the device and/or method may be configured to provide a stimulus at a better than 1 mm lateral range. For example, at a better than 0.2 mm, 0.25 mm, 0.3 mm, 0.35 mm, 0.4 mm, 0.45 mm, 0.5 mm, 0.6 mm, 0.7 mm, 0.8 mm, 0.9 mm, 1 mm, 1.1 mm, 1.2 mm, 1.3 mm, 1.4 mm, or 1.5 mm lateral range.

In exemplary embodiments, the device/method may be configured such that the application of the stimulus benefits from repeatability. For example, the device may be configured to have a better than 5% variability with respect to the properties of the stimuli delivered with the same settings. For example, better than 2%, 3%, 4%, 5%, 6%, 7%, 8%, 9%, or 10% variability. In exemplary embodiments, the variability limitations may apply to one or more of the, deviation from the intended target, size or the droplet, velocity of the droplet, temperature of the droplet, and/or concentration of chemical stimulus within the droplet.

For example, in terms of the stimulus repeatability, the standard deviation between droplet sizes may be between 0.005 μl and 0.025 μl. For example, the standard deviation between droplet sizes may be about 0.005 μl, 0.01 μl, 0.015 μl, 0.02 μl, or 0.025 μl. For example, the standard deviation between droplet sizes may be between 0.005 μl, to 0.02 μl, 0.01 μl to 0.015 μl, 0.01 μl to 0.025 μl 0.015 μl to 0.02 μl, or 0.02 μl, or 0.025 μl.

In particular, having measured the volume of the dispensed droplet 10 times for 4 particular instrument settings, Table 1 below illustrates exemplary results for the average and standard deviation of the ten repeats. Also included is the percentage of standard deviation of the absolute average volume. As can be seen, the precision in these experiments was better for the larger volumes dispensed. Although this may be partly due to the measurement error for the very small volumes.

TABLE 1

| Instrument settings: | | | | | |
|---|---|---|---|---|---|
| Input Pressure | 202 | 202 | 202 | 300 | mbar |
| Distance to Surface | 40 | 40 | 40 | 40 | mm |
| Valve opening period | 48.4 | 3.3 | 18.8 | 18.8 | msec |
| Dispensed Volume | | | | | |
| of 10x repeated | | | | | |
| measurements | | | | | |
| AVG | 0.999 | 0.077 | 0.401 | 0.518 | μl |
| STDEV | 0.016 | 0.005 | 0.013 | 0.021 | μl |
| % STDEV of AVG | 1.6 | 6.9 | 3.2 | 4.1 | % |

In exemplary embodiments, the device may be considered non-invasive. In exemplary embodiments, the device may be configured to avoid issues related to sterilization because it does not contact any part of the subject. In exemplary embodiments, the non-invasive nature of the device and method may assist in reducing the anxiety of the subject.

In exemplary embodiments, the device and/or method may be configured to include the temporal summation of a repeated stimulus on the same location. In exemplary embodiments, the device may be configured to test the spatial resolution of the sensory system by e.g., having two droplets contact the surface of the subject's cornea simultaneously (or substantially simultaneously) with adjustable lateral separation. With two or more nozzles, various combinations of simultaneous or alternate stimulus may be possible.

In exemplary embodiments, various combinations of one or more of the nozzle type, nozzle diameter, operating pressure and/or valve control may be utilized to ensure that droplet with the correct characteristics (e.g., size) is generated and projected onto the ocular surface (e.g., a predetermined location on the ocular surface). For example, dispersion of the droplet before it contacts the ocular surface may interfere with the sensation because, e.g., the eye lid margins are sensitive to even tiny spray droplets, giving false positives with respect to the intended stimulation. In exemplary embodiments, there may also be a visual disturbance as the droplet touches the ocular surface, generating a small ripple in the tear film. In exemplary embodiments, the devices and/or methods, may implement measures to eliminate one or more of these effects.

In exemplary embodiments, possible solutions to address these issues may include presentation of a bright, high contrast image to the contralateral eye for the subject to concentrate on. Other possible solutions include minimizing or reducing the volume of the droplet or increasing the velocity to achieve perceived stimulation, or switching off the illumination shortly before the droplet is projected (e.g., in a randomized manner to assist with the masking of the stimulus).

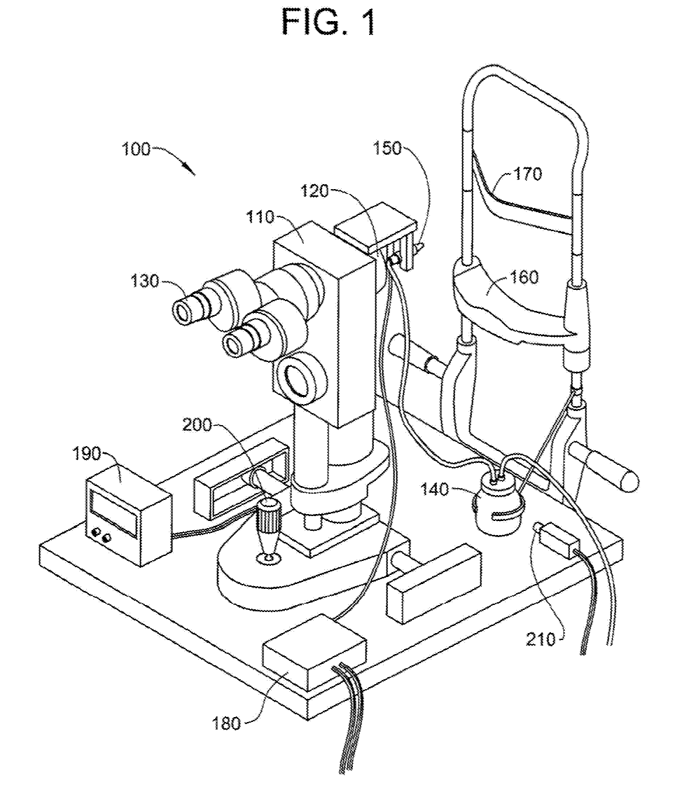

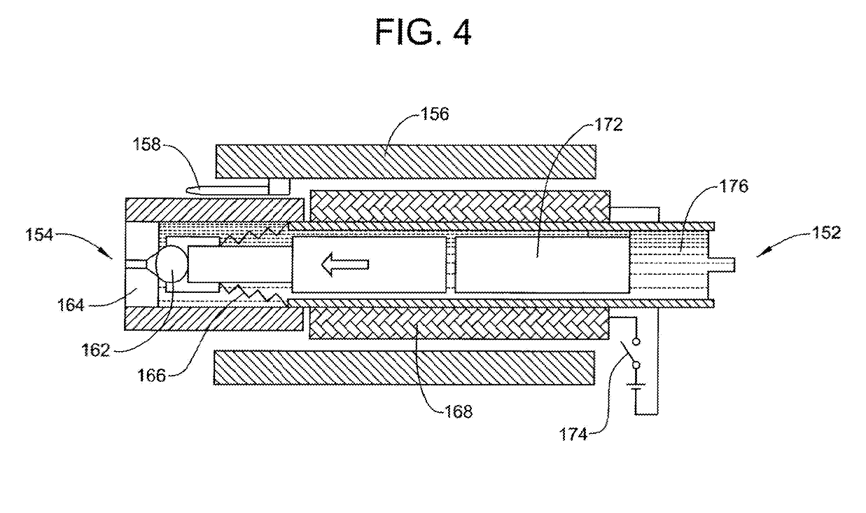

FIG. 1 is an exemplary embodiment of a liquid jet aesthesiometer capable of supplying a stimulus in the form of a liquid droplet to the cornea of a subject. As illustrated, the liquid jet aesthesiometer 100 includes a slit lamp device 110 (or at least slit lamp functionality) configured to illuminate the eye of the subject (light source 120) and provide a view (e.g., a magnified view) of the subject's eye to the individual performing the examination of the eye (binocular view 130). The device includes a liquid reservoir 140 in fluid communication with a nozzle or valve 150 for delivering the liquid stimulus to the eye of the subject when positioned within the chin rest 160 and head rest 170. As illustrated, the device includes circuitry 180 configured to adjust various parameters (e.g., pressure, pulse duration, pulse frequency, pulse delay, etc.) to generate a stimulus with the desired parameters (e.g., size, velocity, etc). The device also includes a temperature controller 190 for controlling the temperature of the liquid delivered to the subject. Although illustrated as a separate device, it should be readily understood that the temperature controller may also be integrated with the control circuitry. In exemplary embodiments, the temperature controller 190 may be operatively coupled to a heating element (or cooling element) for altering the temperature of the liquid. In exemplary embodiments, the heating element (or cooling element) may be located within the valve assembly (see, FIG. 4).

As shown, the device may also include a trigger 200 (e.g., a button) to enable the operator to administer the stimulus to the subject and a feedback button 210 configured to enable the patient to acknowledge whether they were able perceive the stimulus.

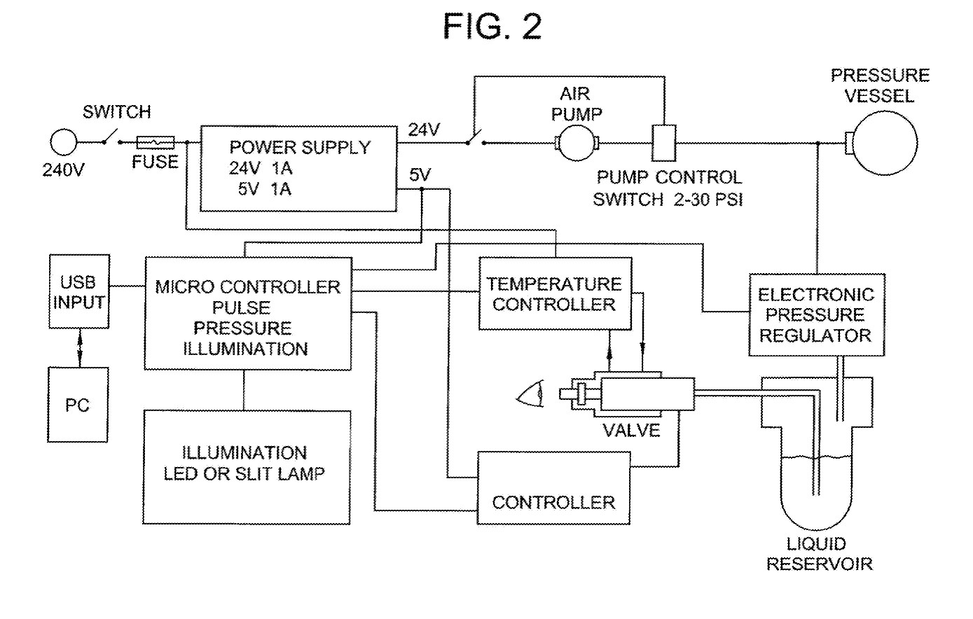

FIG. 2 is block diagram of an exemplary embodiment of a liquid jet aesthesiometer capable of supplying a stimulus in the form of a liquid droplet to the cornea of a subject. As illustrated, the device may include controllers for controlling one or more of the following: pulse, pressure, illumination setting of the device, and the temperature of the fluid.

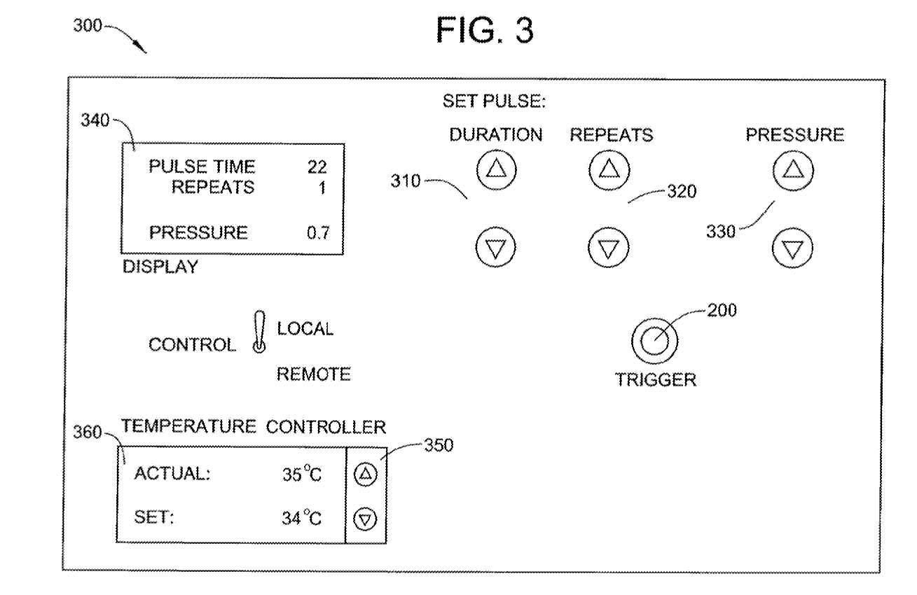

FIG. 3 is an illustration of a control panel for use by an operator for controlling an exemplary liquid jet aesthesiometer. As illustrated in FIG. 3, the control panel 300 may include inputs for adjusting pulse time 310, the number of repeats for pulses 320, and/or the pulse pressure 330. The control panel may also include a display 340 for displaying the value of these parameters. In addition, in exemplary embodiments, the control panel may include inputs for adjusting the temperature 350 of the liquid and a display 360 for displaying the actual and/or set temperature of the liquid. Additionally, although FIG. 1 illustrated a separate mechanical trigger button 200 for initiating the stimulus, in exemplary embodiments, as shown in FIG. 3, the trigger 200 may also be integrated into the control panel. In exemplary embodiments, the device may be connected to and/or controlled by a PC or laptop, or it may be configured to operate as a standalone device with an integrated LCD display and corresponding hardware switches and buttons. Additional control parameters that may be included may be one or more of the following: settings for the delay period between repeated pulses, lateral separation when using two simultaneous droplets, the strength/concentration of a chemical stimulus and timing parameters for switching the illumination on and off. The measurement sequence may be automated. When using the staircase method (described herein), the subject's feedback signal may be used to determine the next stimulus setting, either increasing or decreasing the strength, until several response reversals are achieved and a valid threshold level obtained. In an implementation, this may follow e.g., a one step up on a positive response and two step down on a negative response algorithm. After having obtained a valid sensitivity result, the value for this particular sensitivity test may be compared with normative data to provide some feedback to the practitioner regarding how to interpret the result.

In exemplary embodiments, the instrument may provide the practitioner with information on what is considered a normal measurement result for particular types of stimuli and/or patient groups. This information may be integrated into the software and/or displayed in the user interface or provided in a printed table format for the operator to look up. In exemplary embodiments, normative data for mechanical sensitivity threshold values may be provided for particular areas of ocular surfaces (e.g. central, peripheral cornea), particular tissue types (e.g. cornea, conjunctiva), for a particular patient group (e.g. age, dry eye, Lasik, ocular disease) and/or for a given temperature of the stimulating droplet. Similarly, normative data for chemical and/or thermal threshold values may be provided for particular conditions and/or groups. In exemplary embodiments, the information may provide the practitioner with a reference point to interpret the measured threshold value for an individual subject.

FIG. 4 is an exemplary embodiment of a valve assembly capable of generating and delivering the liquid droplet to the cornea of the subject. As illustrated, the valve 150 includes an inlet 152 for receiving the liquid 176 from the liquid reservoir and an outlet 154 for delivering a droplet to the subject's eye. As illustrated, the exemplary valve assembly further comprises valve ball 162, a valve seat 164, a closing spring 166, a valve coil 168, and a stationary anchor 172. In exemplary embodiments, the valve assemble may be actuated electromagnetically to permit the liquid to flow. For example, in exemplary embodiments, when switch 174 is open and there is no current flowing, the valve may be in a closed position (e.g., the closing spring acts to push the valve ball 162 against the valve seat 164). When the switch 174 is closed, the current may flow through the valve coil 168 to magnetically pull the valve ball and corresponding anchor away from the valve seat 164 and open the valve.

In addition, as discussed herein, the valve includes a heating coil 156 for heating the fluid to a desired temperature prior to being delivered to the subject's cornea (although illustrated as a heating device, the device may be a temperature adjusting device for adjusting the temperature above or below room temperature). The temperature sensor 158 provides feedback to the temperature controller to ensure that the temperature of the valve and liquid remains close to the target temperature.

In exemplary embodiments, nanoparticles, or similar desired size particles, may be utilized either suspended in the liquid or on their own. In exemplary embodiments, various properties of the nanoparticles may be selected to achieve a specific goal (e.g., size color, active coating, etc.)

As discussed herein, in exemplary embodiments, it may be desirable to turn the illumination to the eye off prior to administering the stimulus. For example, it may be desirable to darken the room and/or switch off the target illumination to eliminate, minimize, and/or reduce the optical (visual) sensation for subject as the droplet contacts the tear film and generates a small ripple. In other words, it may be desirable to reduce the potential for false positive results cause by the fact that the subject may see the liquid stimulus. Although FIG. 1 illustrates that the illumination 120 is provided as part of the slit lamp device 110, in exemplary embodiments, the illumination may be provided as part of the aesthesiometer device described herein. For example, in exemplary embodiments, illumination may be provided as part of the structure supporting the nozzle 150. In exemplary embodiments, by including the illumination as part of the aesthesiometer device, it may be possible to control the illumination on the subject's eye, without having to modify the slit lamp (or other device) to turn the illumination off when desired.

In exemplary embodiments, it may be desirable to ask the subject to use headphones or ear plugs to reduce acoustic signals that could influence the subjects' sensation. For example, in some embodiments, when the valve is actuated, there may be a faint but perceivable clicking noise emanating from the valve. Similar to the optical sensation, this may lead to a false positive response from the subject. Such a sound may also give the subject a precise moment when to expect the pulse, making it more difficult to mask for the subject. In addition, the audible triggering when the stimulus strength is controlled by the number of pulses, rather than the length of the valve opening time may enable the subject to directly correlate with the stimulus strength, again taking away the masking for the subject.

In exemplary embodiments, it may be desirable to provide subjects with a button to record their response to the various stimuli. In exemplary embodiments, this may be integrated into the device to facilitate a faster and more objective testing procedure.

As discussed above, the stimulus may be a combination of one or more of mechanical, chemical and/or thermal. For example, in the case of mechanical stimulus, the size and/or velocity of the one or more droplets may be varied. In the case of a thermal stimulus, the temperature for the one or more droplets may be varied. In the case of a chemical stimulus, the pH or other chemical property may be varied (e.g., by the addition or subtraction of compositions to the liquid).

In exemplary embodiments, spluttering may be reduced or controlled by careful selection of valve type, nozzle size (e.g., type and/or diameter) and input pressure.

In exemplary embodiments, various stimulation patterns may be implemented. For example, instead of varying a droplet size, many small droplets of equal size and velocity may be generated in rapid sequence (e.g., 1, 2, 3, or 4 kHz) to increase the stimulus strength. In exemplary embodiments, this may have advantages over having single droplets of varying size or velocity, by reducing spluttering. In addition, a temporal recovery and/or responsiveness may be investigated by e.g., varying the delay time between two or more repeated stimuli. Additional responsiveness may also be evaluated by having two or more valves that shoot out droplets simultaneously, whereby the lateral separation of the droplets may be varied. In exemplary embodiments, similar results may be achieved with one valve that moves quickly between two positions. In exemplary embodiments, different combinations of one or more of these patterns may be implements.

In exemplary embodiments, the sensitivity of various ocular tissues may be investigated (e.g., central or peripheral cornea, limbus, conjunctiva, (everted) eyelid and/or lid margins). In exemplary embodiments, the methods and devices described herein may also be applicable to other surface on the body (e.g., lips, ears, tongue etc.). In exemplary embodiments, the methods and/or devices described herein may have applications in animal research, whereby the natural reflex to stimulations may be one of the indicators.

Although adjustability is useful in the context of the methods and/or devices discussed herein, it may also be useful in exemplary embodiments, to avoid or substantially avoid too many large droplets at rapid sequence, as this may affect the tear volume or composition and/or influence sensitivity. In exemplary embodiments, the injected liquid volume per minute may be less than 1% of the tear volume. For example, the injected liquid volume per minute may be less than 0.2%, 0.4%, 0.6%, 0.8%, 1%, 1.2%, 1.4%, 1.6%, 1.8% or 2% of the tear volume. In exemplary embodiments, droplet sizes may vary between 1 nl to 2 μl. For example, in exemplary embodiments, the droplet sizes may be between about 1 nl to 1 μl, 10 nl to 1 μl, 100 nl to 500 μl, 1 nl to 1.5 μl, 10 nl to 1.5 μl, 100 nl to 1.5 μl, 500 nl to 1.5 μl, 5 nl to 2 μl, 10 nl to 2 μl, 100 nl to 2 μl, 250 nl to 2 μl, or 500 nl to 2 μl. In exemplary embodiment, droplet sizes below about 0.2 nl, 0.4 nl, 0.6 nl, 0.8 nl, 1 nl, 1.2 nl, 1.4 nl, 1.6 nl, 1.8 nl, or 2 nl may be too small. In exemplary embodiment, droplet sizes above about 1 μl, 1.5 μl, 2 μl, 2.5 μl or 3 μl may be too large.

In exemplary embodiments, the velocity of the droplet as it contacts the ocular surface may be between about 0.5 m/s and 5 m/s. For example, the velocity of the droplet as it contacts the ocular surface may be between about 0.5 m/s and 2 m/s, 0.5 m/s and 3 m/s, 0.5 m/s and 4 m/s, 0.5 m/s and 5 m/s, 1 m/s and 5 m/s, 2/s and 5 m/s, 3 m/s and 5 m/s, or 4 m/s and 5 m/s.

In exemplary embodiments, the liquid may be one selected to mimic tear properties. In exemplary embodiments, the liquid may be one selected to have osmolarity similar to that of tears in normal eyes. In exemplary embodiments, the osmolarity of the liquid may not be greater than 295 mOsm/L. For example, the osmolarity of the liquid may be about 270, 275, 280, 285, 290, 295, or 300 mOsm/L. In exemplary embodiments, the liquid may have viscosity that increases the break-up time of the tear film of the eye. In exemplary embodiments, the liquid may be colored.

In exemplary embodiments, the liquid may be degassed to prevent, minimize, and/or reduce air bubbles in the system.

To assist with the masking of the stimulus, in exemplary embodiments, it may be helpful to slightly vary the actual time-point of stimulation with respect to other, earlier stimulation or in relation to the diming of illumination. In exemplary embodiments, this may make it less predictable for the subject to know when to expect the stimulation.

Additionally, synchronizing the stimulus with the subject's blink may be desirable. In exemplary embodiments, optical or acoustical signals may be used to trigger a blink at a pre-determined time period prior to delivering the droplet. In exemplary embodiments, the blink of the subject may be monitored and used as the trigger to deliver the droplet after a certain delay time.

In exemplary embodiments, the device described herein may be attached to a slit lamp to facilitate use while providing accurate targeting of the stimulation spot. The use of a slit lamp may also assist to achieve a repeatable working distance by keeping the surface in focus.

In exemplary embodiments, it may be desirable to provide a working distance of about 10, 20, 30, 40, 50, or 60 mm between the ocular surface and the tip of the nozzle. In exemplary embodiments, it may be desirable to provide a working distance of about 5-60 mm, 10-50 mm, 10-40 mm, 20-40 mm, 30-50 mm, 30-60 mm, or 30-40 mm between the ocular surface and the tip of the nozzle. In exemplary embodiments, these distances may be desired because they reduce anxiety for the subjects. Within a measurement series, it may be desirable to keep a substantially constant working distance to reduce unwanted variability of the stimulus strength.

In exemplary embodiments, active pressure generating devices (used with our without a valve) may be used to eject a liquid droplet (e.g. similar to bubble jet technology or piezo activated printing head technology). In exemplary embodiments, this may eliminate the need for an air pump and pressure control. For example, utilizing this type of method and/or device, the stimulus intensity may be varied by deploying varying numbers of drops in rapid succession.

In exemplary embodiments, a chemical stimulus may be generated by utilizing two ejectors aimed at the same or substantially the same spot on the ocular surface—one ejector may deploy a chemical stimulant and the other may deploy plain water or a neutralizing liquid. By adjusting the relative volume ratio of the two liquids the chemical strength off the stimulus may be varied.

As discussed throughout this specification, various combinations of the described stimuli may be implemented in a manner that is of interest in ophthalmic research as well as e.g., neuroscience.

In exemplary embodiments, the devices and methods described herein may be utilized for precisely quantified topical application of ocular pharmaceutical agents, either in a research setting or for general use. For example, this may be integrated into a spectacle frame, which may make it easier to repeatedly apply very small quantities at regular intervals.

Similarly, in exemplary embodiments, the devices and methods described herein, may provide relief for dry eye patients by e.g., regularly applying wetting agent liquids to the ocular surface, either on demand or at fixed regular intervals. The control circuit may include a sensor to prevent application while the eyelid is closed.

In exemplary embodiments, the devices and methods described herein may be used to measure tear volume. For example, a known amount/concentration of fluorescein, fluorexon, nanoparticles or similar composition may be delivered onto the eye of the subject. This liquid will be diluted by tears and the resulting concentration of the liquid may be proportional to the tear volume.

Animal models using the devices and methods described herein may also be used in different areas of medical research. For example, the devices and methods may be used to obtain feedback through observing blink reflex or through electro-neurological signals. The liquid described herein may include specific pathogens to challenge an infection/inflammation response from a cornea. The devices and methods described herein may be used to apply topical medication. Additionally, by increasing the mechanical stimulus strength to a point where the epithelium is being damaged in a controlled and predictable way may be utilized in the evaluation of corneal wound healing medications. Similarly, precise chemical injuries or burns may be generated using the devices and or methods described herein.

While exemplary embodiments have been shown and described herein, it will be obvious to those skilled in the art that such embodiments are provided by way of example only. It is intended that the following claims define the scope of the invention and that methods and structures within the scope of these claims and their equivalents be covered thereby.

What is claimed is:

1. A device for generating a stimulus in the form of at least one liquid droplet to evaluate ocular sensitivity, the device comprising:
a light source configured to illuminate an eye of the subject;
a liquid reservoir configured to store a liquid;
a nozzle in fluid communication with the liquid reservoir and configured to deliver at least one liquid droplet to at least one eye of a subject;
wherein delivery of the at least one liquid droplet to the eye of the subject provides a stimulus to the ocular surface of the subject's eye and enables the evaluation of the ocular sensitivity of the subject's eye.

2. The device of claim 1, further comprising a slit lamp device (or at least slit lamp functionality) configured to illuminate the eye of the subject and provide a view (e.g., a magnified view) of the subject's eye.

3. The device of claim 1 or 2, further comprising circuitry configured to adjust various parameters (e.g., pressure, pulse duration, pulse frequency, pulse delay, etc.) to generate the at least one liquid droplet such that it possesses the desired parameters (e.g., size, velocity, etc).

4. The device of one or more of the preceding claims, further comprising a temperature controller for controlling the temperature of the at least one liquid droplet delivered to the subject.

5. The device of one or more of the preceding claims, further comprising a heating element (or cooling element) for altering the temperature of the liquid.

6. The device of one or more of the preceding claims, wherein a heating element (or cooling element) is located within the valve assembly.

7. The device of one or more of the preceding claims, wherein the liquid droplet creates a mechanical, chemical, and/or thermal stimulus.

8. The device of one or more of the preceding claims, wherein the liquid is tear-like and/or is warmed up to substantially the same temperature as the eye of the subject.

9. The device of one or more of the preceding claims, wherein the volume of one or more droplets and/or its velocity is adjusted to adjust the strength of the stimulus.

10. The device of one or more of the preceding claims, wherein a sub-mechanical threshold setting is used and the liquid is modified to make it increasingly acidic or alkaline i.e., use of a soap and/or concentrated saline solutions, etc.

11. The device of one or more of the preceding claims, wherein the liquid droplet is heated or cooled while keeping the mechanical stimulation at a sub-threshold level.

12. The device of one or more of the preceding claims, wherein the device is configured to apply the liquid droplet to a precise, predetermined location on the ocular surface of the subject's eye.

13. The device of one or more of the preceding claims, wherein the device is considered non-invasive.

14. The device of one or more of the preceding claims, wherein the device is configured to provide repeated stimulus on the same location.

15. The device of one or more of the preceding claims, wherein device comprises two or more nozzles and the two or more nozzles are configured to provide various combinations of simultaneous or alternate stimulus.

16. The device of one or more of the preceding claims, wherein the device is configured to test the spatial resolution of the sensory system by having two droplets contact the surface of the subject's cornea simultaneously (or substantially simultaneously) with adjustable lateral separation.

17. The device of one or more of the preceding claims, wherein the device is configured to include presentation of a bright, high contrast image to the contralateral eye for the subject to concentrate on.

18. The device of one or more of the preceding claims, wherein the device is configured to reduce/minimize the volume of the droplet and increasing the velocity to achieve perceived stimulation.

19. The device of one or more of the preceding claims, wherein the device is configured to switch off illumination shortly before the droplet is projected (e.g., in a randomized manner to assist with the masking of the stimulus).

20. The device of one or more of the preceding claims, wherein the device further comprises a trigger (e.g., a button) to enable the operator to administer the liquid droplet to the subject.

21. The device of one or more of the preceding claims, wherein the device further comprises a feedback button configured to enable the patient to acknowledge whether they were able perceive the liquid droplet.

22. The device of one or more of the preceding claims, wherein the liquid comprises nanoparticles suspended in the liquid or mixed with the liquid prior to delivery and selected to achieve a specific goal (e.g., size color, active coating, etc.).

23. The device of one or more of the preceding claims, wherein a plurality of droplets of equal size and velocity are generated in rapid sequence (e.g., 1, 2, 3, or 4 kHz) to increase the stimulus strength of the liquid.

24. The device of one or more of the preceding claims, wherein the delay time between two or more repeated stimuli are varied.

25. The device of one or more of the preceding claims, wherein responsiveness is evaluated by having two or more valves that deliver droplets simultaneously and the lateral separation of the droplets may be varied (or alternatively with one valve that moves quickly between two positions).

26. The device of one or more of the preceding claims, wherein the device is configured to avoid delivering too many large liquid droplets at rapid sequence.

27. The device of one or more of the preceding claims, wherein the liquid may be degassed to prevent, minimize, and/or reduce air bubbles in the system.

28. The device of one or more of the preceding claims, wherein the device is configured to slightly vary the actual time-point of stimulation with respect to other, earlier stimulation or in relation to the diming of illumination.

29. The device of one or more of the preceding claims, wherein the stimulus is substantially synchronized with the subject's blink.

30. The device of one or more of the preceding claims, wherein optical or acoustical signals are used to trigger a blink at a pre-determined time period prior (or after) delivering the liquid droplet.

31. The device of one or more of the preceding claims, wherein the device is configured to monitor the blink of the subject and to deliver the droplet after a certain delay time.

32. The device of one or more of the preceding claims, wherein the working distance between the nozzle and the ocular surface is about 10, 20, 30, 40, 50, or 60 mm.

33. The device of one or more of the preceding claims, wherein active pressure generating devices (used with our without a valve) may be used to eject a liquid droplet (e.g. similar to bubble jet technology or piezo activated printing head technology).

34. The device of one or more of the preceding claims, wherein a chemical stimulus is generated by utilizing two ejectors aimed at the same spot on the ocular surface—one ejector configured to deploy a chemical stimulant and the other injector configured deploy plain water or a neutralizing liquid, in a predefined ratio.

35. A method for evaluating ocular sensitivity, the method comprising:
storing a liquid in a liquid reservoir;
transmitting the liquid from the liquid reservoir to a nozzle;
generating at least one liquid droplet; and
delivering the at least one liquid droplet to the ocular surface of a subject's eye;
wherein the delivery of the at least one liquid droplet to the eye of the subject provides a stimulus to the ocular surface of the subject's eye and enables the evaluation of the ocular sensitivity of the subject's eye.

36. The method of claim 35, further comprising providing a light source configured to illuminate an eye of the subject;

37. The method of claim 35 or 36, further comprising adjusting various parameters (e.g., pressure, pulse duration, pulse frequency, pulse delay, etc.) to generate the at least one liquid droplet such that it possesses the desired parameters (e.g., size, velocity, etc).

38. The method of one or more of claims 35-37, further comprising controlling the temperature of the at least one liquid droplet delivered to the subject.

39. The method of one or more of claims 35-38, further comprising heating (or cooling) the liquid.

40. The method of one or more of claims 35-39, wherein the liquid is heated within the valve assembly.

41. The method of one or more of claims 35-40, wherein the liquid droplet creates a mechanical, chemical, and/or thermal stimulus.

42. The method of one or more of claims 35-41, wherein the liquid is tear-like and/or is warmed up to substantially the same temperature as the eye of the subject.

43. The method of one or more of claims 35-42, further comprising adjusting the volume and/or velocity of the liquid droplets to adjust the stimulus.

44. The method of one or more of claims 35-43, reducing the delivery of the liquid droplet to a sub-mechanical threshold and modifying the liquid to make it increasingly acidic or alkaline i.e., use of a soap and/or concentrated saline solutions, etc.

45. The method of one or more of claims 35-44, further comprising heating or cooling the liquid droplet while keeping the mechanical stimulation at a sub-threshold level.

46. The method of one or more of claims 35-45, further comprising applying the liquid droplet to a precise, predetermined location on the ocular surface of the subject's eye.

47. The method of one or more of claims 35-46, wherein the method is considered non-invasive.

48. The method of one or more of claims 35-47, further comprising providing repeated stimulus on the same location of the ocular surface.

49. The method of one or more of claims 35-48, wherein two or more nozzles and the two or more nozzles are configured to provide various combinations of simultaneous or alternate stimulus.

50. The method of one or more of claims 35-49, testing the spatial resolution of the sensory system by having two droplets contact the surface of the subject's cornea simultaneously (or substantially simultaneously) with adjustable lateral separation.

51. The method of one or more of claims 35-50, further comprising presenting a bright, high contrast image to the contralateral eye for the subject to concentrate on.

52. The method of one or more of claims 35-51, further comprising reducing/minimizing the volume of the droplet or increase the velocity to achieve perceived stimulation.

53. The method of one or more of claims 35-52, further comprising switching off illumination shortly before the droplet is projected (e.g., in a randomized manner to assist with the masking of the stimulus).

54. The method of one or more of claims 35-53, further comprising enabling the operator to administer the liquid droplet to the subject.

55. The method of one or more of claims 35-54, further comprising enabling the subject to acknowledge whether they were able perceive the liquid droplet.

56. The method of one or more of claims 35-55, further comprising suspending nanoparticles in the liquid or mixing the nanoparticles with the liquid prior to delivery to achieve a specific goal (e.g., size color, active coating, etc.).

57. The method of one or more of claims 35-56, further comprising generating a plurality of droplets of equal size and velocity in rapid sequence (e.g., 1, 2, 3, or 4 kHz) to increase the stimulus strength of the liquid.

58. The method of one or more of claims 35-57, further comprising varying the delay time between two or more repeated stimuli.

59. The method of one or more of claims 35-58, further comprising evaluating responsiveness by having two or more valves that deliver droplets simultaneously such that the lateral separation of the droplets may be varied (or alternatively with one valve that moves quickly between two positions).

60. The method of one or more of claims 35-59, further comprising avoiding delivering too many large liquid droplets at rapid sequence.

61. The method of one or more of claims 35-60, further comprising degassing the liquid to prevent, minimize, and/or reduce air bubbles in the system.

62. The method of one or more of claims 35-61, further comprising varying the actual time-point of stimulation with respect to other, earlier stimulation or in relation to the diming of illumination.

63. The method of one or more of claims 35-62, wherein the stimulus is synchronized with the subject's blink.

64. The method of one or more of claims 35-63, further comprising triggering a blink using optical or acoustical signals at a pre-determined time period prior (or after) delivering the liquid droplet.

65. The method of one or more of claims 35-64, further comprising monitoring the blink of the subject and delivering the droplet after a certain delay time.

66. The method of one or more of claims 35-65, wherein the working distance between the nozzle and the ocular surface is about 10, 20, 30, 40, 50, or 60 mm.

67. The method of one or more of claims 35-66, further comprising generating a chemical stimulus by utilizing two ejectors aimed at the same spot on the ocular surface—one ejector configured to deploy a chemical stimulant and the other injector configured deploy plain water or a neutralizing liquid, in a predefined ratio.
